(19) United States
(12) Patent Application Publication
(10) Pub. No.: US 2017/0067088 A1
(43) Pub. Date: Mar. 9, 2017

(54) CATIONIC POLYMER SYSTEMS FOR SELECTIVE BACTERIAL CAPTURE

(71) Applicant: THE JOHNS HOPKINS UNIVERSITY, BALTIMORE, MD (US)

(72) Inventors: DEVIN O'BRIEN-COON, BALTIMORE, MD (US); ANJANA SINHA, PENNINGTON, NJ (US); YUKA MANABE, BALTIMORE, MD (US); GYANU LAMICCHANE, TOWSON, MD (US); HECTOR NEIRA, BURTONSVILLE, MD (US); HAI-QUAN MAO, BALTIMORE, MD (US); HIREN MISTRY, BALTIMORE, MD (US); ANMOL CHOPRA, FARIDABAD, HARYANA (IN); XUESONG JIANG, LUTHERVILLE, MD (US)

(73) Assignee: THE JOHNS HOPKINS UNIVERSITY, BALTIMORE, MD (US)

(21) Appl. No.: 15/308,139

(22) PCT Filed: May 6, 2015
(86) PCT No.: PCT/US15/29498

§ 371 Date: Nov. 1, 2016

Related U.S. Application Data
(60) Provisional application No. 61/989,296, filed on May 6, 2014.

(51) Int. Cl. C12Q 1/04; C12Q 1/18; C12Q 1/24

(52) U.S. Cl. CPC C12Q 1/04; C12Q 1/24; C12Q 1/18; G01N 2333/35; G01N 2500/10; G01N 2650/00

(57) ABSTRACT

A device and methods of use thereof for isolating one or more microorganisms from a biological sample, the device comprising a polymeric surface having one or more cationic polymers covalently grafted thereto, wherein the one or more cationic polymers have a selective affinity for the one or more microorganisms.

### CROSS-REFERENCE TO RELATED APPLICATIONS

This application claims the benefit of U.S. Provisional Application No. 61/989,296, filed May 6, 2014, which is incorporated herein by reference in its entirety.

### BACKGROUND

Tuberculosis is a major global health problem. The World Health Organization (WHO) estimated that there were 8.6 million new cases of tuberculosis (TB) and 1.3 million TB-related deaths in 2012. WHO. Global Tuberculosis Report 2013. TB is an airborne infectious disease caused by the bacterium Mycobacterium tuberculosis (MTB) and is concentrated primarily in the developing world. It is estimated that only 66% of the incident TB cases globally are notified to TB control programs. Further, of the 4.6 million pulmonary cases reported globally in 2012, only 57% were bacteriologically confirmed. Accordingly, diagnosing the more than 3 million missed cases of TB is a WHO priority.

To prevent transmission of TB in the community, to provide appropriate care for patients, and to reduce the burden of the disease, prompt and accurate diagnosis of TB is a matter of great urgency. Nunn et al., Nat Rev Immunol. (2005). Rapid, reliable, and accessible technology suitable for resource-limited settings is needed to increase the proportion of TB patients who are diagnosed and referred for available treatment for a curable disease. The lack of an affordable tuberculosis (TB) diagnostic that can be deployed in rural settings with improved accuracy over sputum smear microscopy remains a significant stumbling block to TB control.

In low-income and middle-income countries, where 95% of TB cases and 98% of deaths occur, direct sputum smear light microscopy is the primary method for diagnosing pulmonary TB. This 125-year old method is fast, inexpensive, and widely available at peripheral facilities of high incidence countries. Direct microscopy has low sensitivity, however, and diagnoses only 20-60% of patients who have active TB with a single smear. Getahun et al., Lancet. (2007). The sensitivities are lowest in individuals co-infected with HIV and in pediatric patients. A reason for this low sensitivity is that the microscopic limit of detection is 5,000-10,000 bacteria/mL. Furthermore, the concentration of bacteria in a sputum sample is not uniform, so the sensitivity depends on the particular portion of the specimen sampled by the direct microscopic measurement.

Fluorescence microscopy (FM) using Auramine O staining for the detection of mycobacteria has been used for decades, Hagemann, Zentralbl Bakteriol (1937), but has been limited by the mercury vapor lamp (MVL) technology used in conventional FM, which has an expensive power supply, is inefficient, short-lived and has the potential to release toxic mercury. Anthony, et al., Int J Tuberc Lung Dis. (2006). FM, however, is approximately 10% more sensitive than conventional light microscopy (LM) using Ziehl-Neelsen staining. Steingart et al., Scand. J. Respir. Dis. (1966). FM also is more efficient because the staining protocol is more efficient, slides can be read at lower magnification and requires a shorter examination time per slide. Bennedsen and Larsen, Scand. J. Respir. Dis. (1966).

Enhancements have been made to the conventional sputum smear microscopy by utilizing light-emitting diodes (LED) for fluorescent microscopy. Although this approach improves sensitivity (5-6% overall), Trusov et al., Int J Tuberc Lung Dis. (2009); Van Deun et al., Int J Tuberc Lung Dis. (2008); WHO. Fluorescent light emitting diode microscopy for diagnosis of TB (2010), it does not address the root cause of the problem, the minimal capture of MTB from the sample. Steingart, et al., Lancet Infect Dis. (2006).

MicroSens (Lowell, Mass.) has created magnetic bead technology in an attempt to concentrate the MTB in sputum prior to microscopy. U.S. Patent Application Publication No. US2010/0143883 A1, for Capture of Mycobacteria Like Micro-Organisms, to Wilson et al., published Jun. 10, 2010, which is incorporated herein by reference in its entirety. The product adds significant cost to a diagnostic test that otherwise costs cents, and adds more steps and biohazards to the workflow, which requires cumbersome modifications to the infrastructure. Despite advances in microscopy such as LED, the sensitivity of sputum smear microscopy is still limited by the relatively small amount of sputum that can be put on the glass slide, which represents only a fraction of the bacteria in the patient's lungs.

The gold standard for TB diagnosis remains culture, either solid or liquid. All culture methods require biosafety level 3 (BSL-3) laboratories because the sputum decontamination processing includes centrifugation, which increases the risk for aerosol generation. Without BSL-3 facilities, these procedures would pose a significant occupational risk to laboratory personnel. Unfortunately, very few BSL-3 facilities exist in TB high burden countries as they are expensive to build and maintain.

In 2011, the WHO recommended Xpert MTB/RIF (Cepheid, Sunnyvale, Calif.) for diagnosis of pulmonary TB and rifampicin resistance in adults. It is the first rapid molecular test that can be used to simultaneously test for TB and rifampicin resistance, with 98% sensitivity in sputum smear positive patients and sensitivity that ranges from 55-72% in a single sputum from smear-negative patients. Boehme et al., Lancet. (2011). Despite its promise as a rapid molecular test, there have been operational challenges, which include the requirement for an ambient temperature of lower than 30° C. (necessitating air conditioning in hot climates), and uninterrupted and stable electrical power supply (requiring generators in several sites). When users were queried, storage space and conditions (28° C.) for cartridges, waste generated (considerably more than for microscopy), and the 12-month shelf-life of cartridges were listed as the main operational challenges. Weyer et al., Eur Respir J. (2013). An initial capital investment (machine and computer and approximately $17,000) is required along with on-going maintenance costs. Accordingly, currently available TB diagnostics that have higher sensitivity than sputum smear microscopy are expensive, require new infrastructure development and their widespread adoption is not expected in the near future.

### SUMMARY

In some aspects, the presently disclosed subject matter provides a device for isolating one or more microorganisms from a biological sample, the device comprising a polymeric surface having one or more cationic polymers covalently grafted thereto, wherein the one or more cationic polymers have a selective affinity for the one or more microorganisms. In particular aspects, the cationic polymer comprises poly-diallyldimethyl ammonium chloride (pDADMAC).

In other aspects, the presently disclosed subject matter provides a method for isolating one or more microorganisms from a biological sample, the method comprising: (a) providing a device comprising a polymeric surface having one or more cationic polymers covalently grafted thereto, wherein the one or more cationic polymers have a selective affinity for the one or more microorganisms; (b) contacting the biological sample with the polymeric surface having one or more cationic polymer covalently grafted thereto to bind the one or more microorganisms to the polymeric surface; (c) staining the bound microorganisms; and (d) analyzing the stained bound microorganisms to determine the presence or absence of the one or more microorganisms in the biological sample.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1 is a schematic workflow for the presently disclosed sputum microscopy device. Left: the sputum is captured into a cup with the TB-capture slide integrated within. After repeated mixing inversions, the slide with the adhered MTB is removed, and then stained and visualized using standard microscopy methods;

FIG. 2a and FIG. 2b are (a) schematic of pDADMAC surface conjugation onto plastic surface, illustrating the presently disclosed plasma-activation step and the UV-initiated grafting polymerization step; and (b) positive charge density grafted onto polystyrene surface initiated under different UV treatment times (0.5, 1, 2 and 3 min.), when reacted with DADMAC monomer solution (n=4);

FIG. 3a, FIG. 3b, FIG. 3c, FIG. 3d, FIG. 3e and FIG. 3f demonstrate the bacteria-capture efficiency for PDADMAC-grafted slides as a function of surface charge density (UV exposure time), bacteria concentration, and bacterial sample volume applied. Current detection limit for smear test is at 10,000 bacilli/mL concentration;

FIG. 4a, FIG. 4b, FIG. 4c, FIG. 4d, FIG. 4e and FIG. 4f are fluorescence images contrasting the uniformity of bacilli distribution on surface between the smear method (drop drying on glass or unmodified plastic surface, (FIG. 4a, FIG. 4b, and FIG. 4c) and the presently disclosed slide-based capturing method (FIG. 4d and FIG. 4e) at 200,000 bacilli/mL (FIG. 4a and FIG. 4d), 50,000 bacilli/mL (FIG. 4b and FIG. 4e) and 10,000 bacilli/ml (FIG. 4c and FIG. 4f) concentration;

FIG. 5a, FIG. 5b, FIG. 5c, FIG. 5d and FIG. 5e show bacteria-capturing efficiency of various pDADMAC-grafted surfaces (treated with different UV-initiation time, see FIG. 2a and FIG. 2b for details) when using 200 μL sample containing 50,000 bacilli/mL: FIG. 5a is a controlled polystyrene (PS) surface without modification; FIG. 5b, FIG. 5c and FIG. 5d are PS surfaces grafted with pDADMAC following 0.5, 1 and 2 min of UV treatment, respectively; and FIG. 5e shows the average bacilli number captured by modified surface and pristine PS surface following sample incubation and then washing with water under shaking for 3 min (n=4); and

FIG. 6 is a comparison of the presently disclosed slide-based bacteria capturing efficiency with that of a smear test for low concentration of bacilli (10,000/mL). The maximum detection limit (100%) for the smear test is indicated with a dotted line. With only two times of the sample volume, the new surface can significantly improve the detection efficiency. The intermediate charge density is optimal. Bars represent mean±standard deviation (n=4).

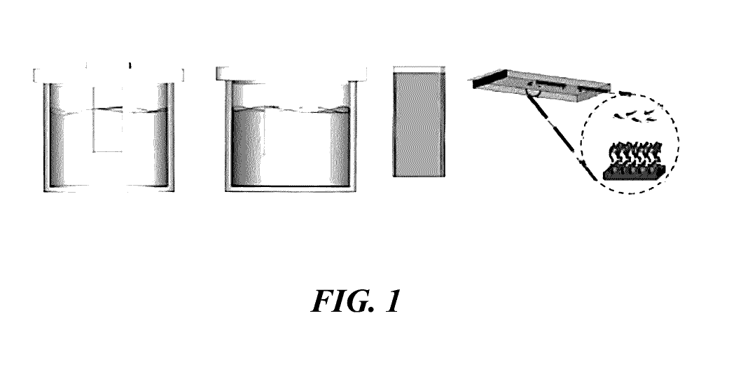

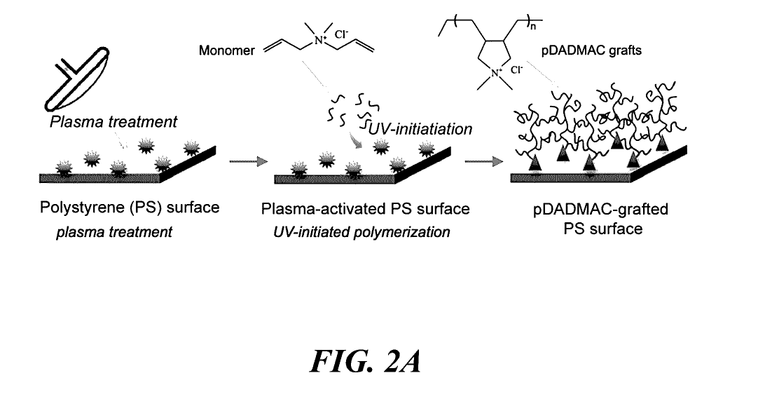

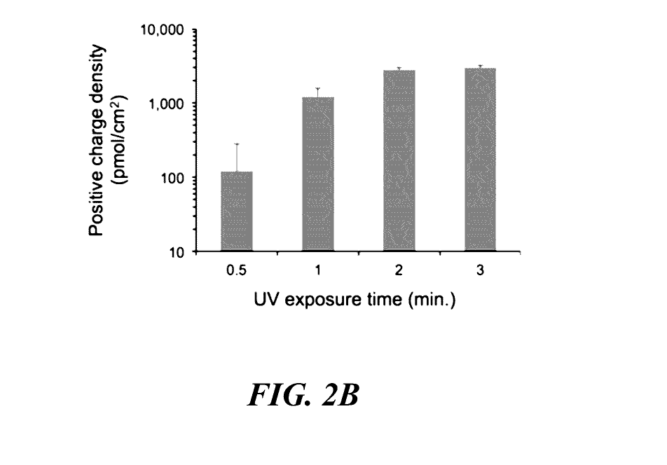

The patent or application file contains at least one drawing executed in color. Copies of this patent or patent application publication with color drawings will be provided by the Office upon request and payment of the necessary fee.

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Polymer-Based System for the Capture of Mycobacteria

Pathogenic mycobacteria are responsible for several severe infectious diseases in humans and animals. Mycobacteria are characterized by a hydrophobic, waxy coat comprising mycolic acid and related compounds. Mycolic acids are complex hydroxylated branched chain fatty acids, typically having hydrocarbon chains with a chain length in the range C₇₇-C₈₀. The waxy coat of pathogenic mycobacteria causes difficulties in sample handling.

Pathogenic mycobacteria include Mycobacterium tuberculosis, which is the causative agent of TB, the mycobacteria of the Mycobacterium avium complex (MAC) complex (primarily M. avium and M. intracellulare), which are opportunistic pathogens in AIDS patients, M. paratuberculosis, which causes bowel inflammation, M. leprae causing leprosy, M. kansasii, M. marinum, M. fortuitum complex, and the like. Other non-pathogenic mycobacteria, include M. smegmatis. Members of the Mycolata family also have similar hydrophobic waxy coats.

An inexpensive, simple, point-of-care diagnostic test for pulmonary tuberculosis has been highly sought after for years. The current state of the art involves the use of various sputum preparations on unmodified glass slides. This technique, however, suffers from a low sensitivity and is not able to diagnose pulmonary tuberculosis infections having a low bacterial count.

To diagnose mycobacterial infections, such as tuberculosis, the presence of the organism is determined by one of several diagnostic tests, including microscopy, culture, or molecular methods, such as PCR. Although microscopy can be done directly from the biological sample, the mycobacteria from the biological specimens are typically isolated and concentrated prior to analysis.

Biological samples containing or suspected of pathogenic mycobacteria include sputum, urine, blood, bronchial lavage, and the like. One of the most common samples used for diagnosing TB is sputum. Sputum, however, presents unique problems for bacteriology. Sputum is heterogeneous in nature and can be bloody, purulent, and viscous. It also can be contaminated with other micro-organisms, for example, Pseudomonas.

Prior to analysis, sputum typically is thinned and decontaminated by various pre-treatments steps, which include the use of 0.25-0.5 M sodium hydroxide with or without N-acetyl L-cysteine, sodium dodecyl sulphate, oxalic acid, or trisodium phosphate. Treatment times can be about 20 minutes to about 120 minutes. Such treatments are designed to thin the sputum and kill the majority of contaminating organisms. Because mycobacteria have a thick waxy coat, they are more resistant to such treatments. Even so, it is estimated that up to 60% of M. tuberculosis are killed or rendered non-viable by this treatment. Further processing of the sample, such as centrifugation, can increase the time and cost of the diagnosis and further risk contaminating the sample or exposing the laboratory technician to the pathogen.

Accordingly, it would be beneficial to be able to selectively isolate the pathogenic mycobacteria directly from the biological sample and, in the process, removing some or all of the contaminating organisms without resorting to harsh chemical decontamination processes. Such a process also would enhance survival of the mycobacteria of interest and increase the sensitivity of the subsequent diagnostic test.

U.S. Patent Application Publication No. US2010/0143883 A1, for CAPTURE OF MYCOBACTERIA LIKE MICRO-ORGANISMS, to Wilson et al., published Jun. 10, 2010, describes physically coating a polar polymer onto glass and allowing it to dry via polar interactions. This physical coating method, however, is not effective in retaining the polymer on the glass surface and for capturing bacteria, if any. In contrast, the presently disclosed subject matter demonstrates that covalent bonding is necessary to reliably capture mycobacteria to a solid surface. Accordingly, the presently disclosed methods provide high efficiency TB capture and retention.

Further, prior work using pDADMAC was not based on the unique characteristics of mycobacteria, but rather on the fact that it is cationic and readily available. Bernhardt and Clasen (1991); Melo et al. (2010). The presently disclosed subject matter demonstrates, however, that systematic engineering and screening of various polymer structures is important for identifying the best device candidates for TB capture with greater sensitivity and specificity. Thus, other compounds based on the chemical properties identified herein also could have the ability to capture mycobacteria. More particularly, as provided in more detail herein below, an intermediate charge density and moderate hydrophobicity are characteristic of the optimized surfaces. When the polymer is hydrophilic, pDADMAC-grafting will not give good TB capturing efficiency.

In developing such a diagnostic test for TB and other mycobacteria for developing countries and regions, four critical criteria must be considered. The test must be: based on a low-cost platform since the disease is concentrated in low- and middle-income countries; capable of enriching MTB from sputum samples, thereby increasing detection sensitivity; self-contained to minimize risk of contamination and the number of transfers of sputum samples between containers to reduce the biohazard risk; and compatible with existing microscope technologies to reduce infrastructure requirements.

One goal of the presently disclosed subject matter is to improve the sensitivity of sputum microscopy, thereby allowing hundreds of thousands of additional new cases of tuberculosis to be diagnosed and referred for treatment each year. One target goal of the presently disclosed subject matter is to exceed the improvement made by fluorescence LED microscopy, which has recently been endorsed by the WHO but offers only a 5-6% increase in sensitivity with an additional requirement for procurement of new equipment (LED microscope). The presently disclosed methods could achieve such an impact with minimal disruption to current workflow, which enables the presently disclosed methods to be easily deployed and implemented.

Further, the presently disclosed methods provide increased bacterial recovery to improve diagnostic sensitivity without the high cost, additional equipment, and cumbersome procedures found in methods known in the art. The presently disclosed methods also can be combined with LED microscopy to further increase its detection sensitivity.

Accordingly, the presently disclosed subject matter provides a polymeric system for capturing mycobacteria, for example, tuberculosis bacteria, thereby permitting a more sensitive diagnosis of TB. More particularly, the presently disclosed subject matter provides slides or films modified with a polymer having an affinity for particular mycobacteria that can selectively bind the mycobacteria on a surface, thereby enriching the mycobacteria of interest present within a biological sample and improving the detection limit. Such devices and methods concentrate and/or further manipulate the organism, such as capturing and washing the mycobacteria to remove non-infecting organisms or contaminants or to capture and transfer the mycobacteria from one solution to another.

More particularly, the presently disclosed subject matter provides surface-grafted polycationic polymer chains having an affinity for mycobacteria. In some embodiments, the presently disclosed subject matter provides a series of polymer grafting compositions designed to mimic the structures of tuberculosis bacteria-specific dyes. The non-specific surface properties of charge and hydrophobicity of the cationic polymer can be optimized to distinguish mycobacteria from other organisms found within sputum. The presently disclosed polymer-grafted surfaces exhibit various degrees of mycobacterial affinity and can be used for bacterial enrichment and detection.

In some embodiments, the preparation of the presently disclosed slides or films involve surface grafting of cationic polymer chains onto a polymeric, e.g., plastic, slide or film using a free radical polymerization after plasma activation of the plastic surface. In such polymeric slides or films, a polymer capable of selectively binding mycobacteria of interest is covalently conjugated to the slide or film. In particular embodiments, the polymer is covalently grafted onto a surface of the polymeric slide or film. The polymeric slide or film can be prepared from polymers including, but not limited, to poly(ethylene terephthalate) (PET), polystyrene (PSt), polyethylene (PE), and poly(methyl methacrylate) (PMMA). The presently disclosed slides or films are prepared, in some embodiments, by using UV-grafting techniques to provide a solid surface for capturing and enriching mycobacteria, for example, tuberculosis bacteria, from sputum samples.

In particular embodiments, the presently disclosed device includes an optimized poly(dimethyl diallyl chloride) (pDADMAC)-grafted plastic slide platform, wherein pDADMAC is:

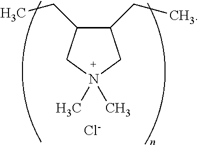

The molecular weight of the pDADMAC may be in the range of less than 100,000 (very low), 100,000-200,000 (low), 200,000-400,000 or 500,000 (medium) or over 500,000 (high).

MTB bacteria can be effectively captured by the presently disclosed pDADMAC-grafted slides with higher sensitivity than the maximum smear test known in the art (as provided herein below). This characteristic allows for a diagnostic test to be created that harnesses higher sensitivity, while remaining low-cost, robust with no additional infrastructure needed in the developing world.

The presently disclosed polycationic polymers, including pDADMAC, can be used in a variety of embodiments for the capture of mycobacteria. In one embodiment, a slide is prepared that can be used for sputum microscopy as is currently performed, but with the advantage of capturing more bacteria for diagnosis. Current methods place a small portion of the sample (<0.1 mL) onto the slide, which means that most of the bacteria in the sample are lost. By having a slide that captures bacteria, more of the sample can be brought into contact with the slide, thereby increasing bacterial count and consequently sensitivity.

Slides prepared in this way can be characterized via staining and demonstrate a reliable, reproducible process for producing the coating. Additional methods are available to further optimize interaction with the sputum sample (i.e., dipping, swirling, and the like). Other uses for the polymeric coating may include capture for culture growth and drug sensitivity testing.

The presently disclosed diagnostic test for TB can be easily incorporated into the existing workflow for sputum microscopy and the end user continues to be the technologist at the sputum microscopy center. In particular embodiments, a unique sputum cup, housing the presently disclosed pDADMAC-grafted plastic slide, is used to collect the sputum sample. After diluting with a solution to decrease viscosity and increase volume, this slide is exposed repeatedly to the MTB within the sample by performing multiple mixing inversions. Due to the slide's high affinity for MTB, a large proportion of MTB in the sample is then captured onto a fixed area on the slide, thereby enriching MTB. The MTB adhered on the slide can then be stained with either Ziehl-Neelsen or Auramine O to be incorporated into basic light microscopy or LED fluorescence microscopy. Visual assessment of the sample is more comprehensive and more sensitive than existing methods as a higher proportion of MTB from the patient's original sputum sample is examined.

Also, in further embodiments, closed systems built using the presently disclosed technology (e.g., for culture and multi-drug resistance detection) can include features including, but are not limited to, the geometry of the mycobacterial capture area, the form factor, design and method of use of the sputum microscopy system and self-contained MDR-TB assay, and the use of a non-fluorescent growth detection marker for the MDR-TB assay.

The subject treated by the presently disclosed methods in their many embodiments is desirably a human subject, although it is to be understood that the methods described herein are effective with respect to all vertebrate species, which are intended to be included in the term “subject.”

A “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The descriptions and specific examples that follow are only intended for the purposes of illustration, and are not to be construed as limiting in any manner to make compounds of the disclosure by other methods.

### Example 1

### Grafting and Characterization of pDADMAC on Plastic Surface

Surface-grafting of pDADMAC on both polyethyleneterephalate (PET) and polystyrene (PS) surfaces was accomplished. PET and PS are common plastic materials and easily obtained for processing, although PET has a higher fluorescence background. In some embodiments, the presently disclosed surface grafting method includes a plasma treatment step to activate the plastic surface, followed by a UV-initiated free radical polymerization step to graft pDADMAC onto the plastic surface in a reliable and consistent manner. After screening various parameters, including temperature, monomer concentration, plasma treatment time, UV treatment time, polymerization time, monomer types, and the like, UV treatment time was identified as the most effective means to adjust charge density on the surface. Using an established calibration curve, it was demonstrated that a positive charge density in the range of 0.1-4 nmol/cm² can be achieved consistently. The pDADMAC grafts are stable when incubated in culture media over a period of at least 15 hours. The pDADMAC-grafted surfaces are stable at room temperature with a measured shelf-life of at least 2 months.

### Example 2

### Optimization of Bacteria Capture Efficiency on pDADMAC-Grafted Plastic Surfaces

Using a non-pathogenic, fluorescently tagged surrogate mycobacterial strain, the bacteria-capture efficiency of various pDADMAC-grafted surfaces was tested. These tests confirmed that pDADMAC-grafted surfaces can effectively capture and enrich Mycobacterium. These tests also quantified important factors in determining bacterial capture efficiency: (1) the positive charge density on the modified surface; (2) the bacterial concentration in the sample; and (3) sample volume.

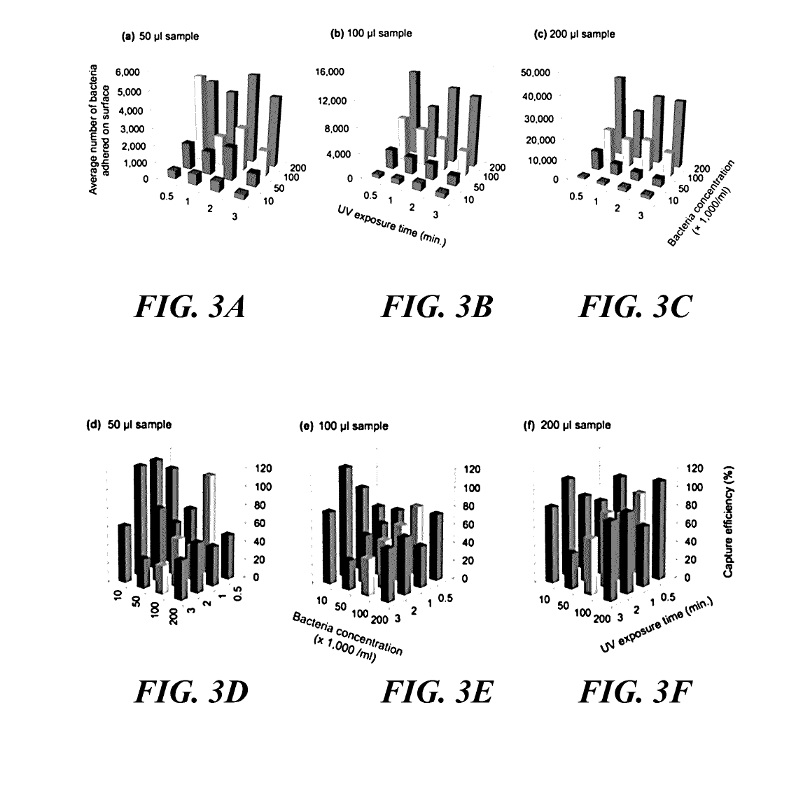

Referring now to FIG. 3 a representative set of data highlighting these relationships is shown. These data suggest that intermediate charge density (approximately 1 nmol/cm²) is optimal for capturing bacilli from a suspension, which was an unexpected result considering that the highest charge density would have been expected to be optimal for capturing bacilli. Interestingly, the highest concentration of positive charge does not increase bacteria-capture efficiency, particularly at low concentration of bacilli. One advantage is the drastically improved bacterial distribution on the presently disclosed slides, particularly at medium and low concentration of bacilli in samples.

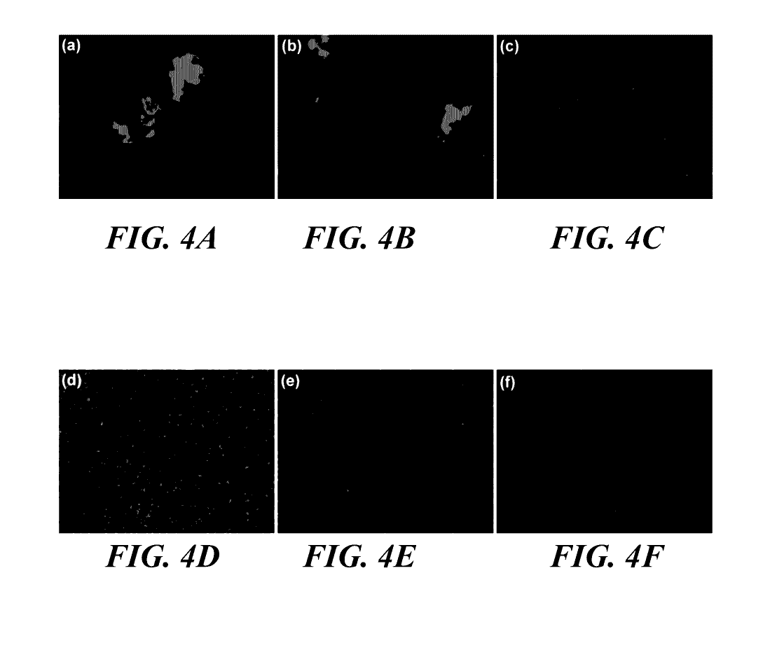

Referring now to FIG. 4, it is demonstrated that a traditional smear test will likely result in uneven distribution of the bacilli and cause difficulty in manual counting. In contrast to methods known in the art, the presently disclosed pDADMAC-grafted surface yields more uniform distribution of the bacilli.

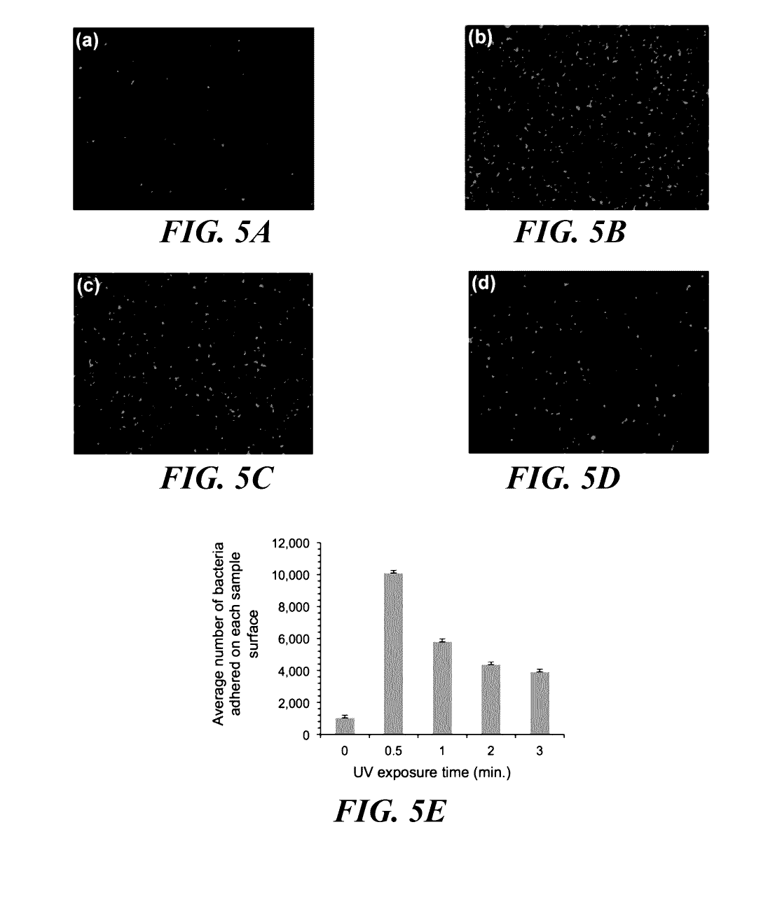

Further, one surprising finding is that optimal bacteria capture efficiency requires low to intermediate charge density on the plastic surface (see FIG. 5). This range varies depending on the materials used for pDADMAC grafting. Without wishing to be bound to any one particular theory, these results suggest that a balanced positive charge and hydrophobicity is crucial to achieving high capture efficiency and retention.

### Example 3

### Efficacy of Mycobacterium Capture in Sputum

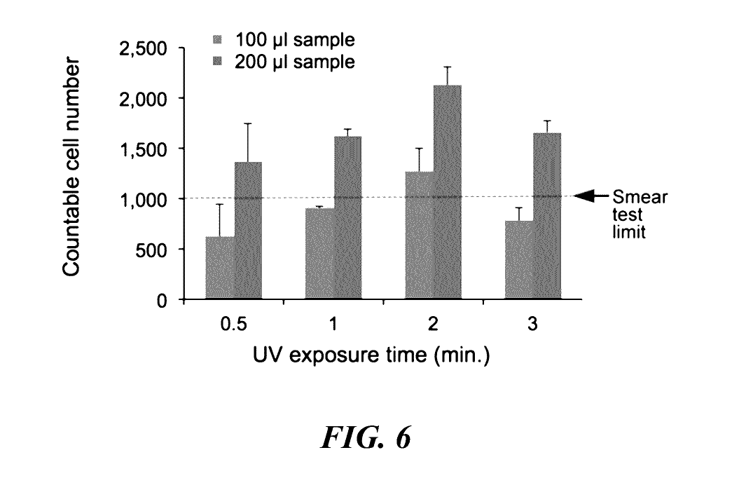

The sensitivity of the presently disclosed bacteria capture surface also was compared to that of a smear test known in the art. Using a sample having a low bacteria concentration (10,000 bacilli/mL), it was shown that the presently disclosed pDADMAC-grafted surfaces have a higher sensitivity, particularly at intermediate surface-charge density (FIG. 6). When using twice the amount of sample volume, the number of bacilli captured by the pDADMAC-grafted surface nearly doubled, indicating approximately 100% bacteria capture efficiency. Preliminary evaluation showed in a laboratory in a developing country indicated an increased Mycobacterium capture as compared to glass slide controls upon repeated exposure to unprocessed sputum (the most challenging sample type).

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
WHO. Global Tuberculosis Report 2013. 2013 [cited Oct. 31, 2013]; Available from: http://www.who.int/tb/publications/global_report/en/
Nunn P, Williams B, Floyd K, Dye C, Elzinga G, Raviglione M. Tuberculosis control in the era of HIV. Nat Rev Immunol. 2005; 5(10): 819-26.
Getahun H, Harrington M, O'Brien R, Nunn P Diagnosis of smear-negative pulmonary tuberculosis in people with HIV infection or AIDS in resource-constrained settings: informing urgent policy changes. Lancet. 2007; 369(9578): 2042-9.
Hagemann P. Fluoreszenzmikroskopische untersuchungen uber virus and andere microben. Zentralbl Bakteriol 1937; 140: 184.
Anthony R M, Kolk A H, Kuijper S, Klatser P R. Light emitting diodes for auramine O fluorescence microscopic screening of Mycobacterium tuberculosis. Int J Tuberc Lung Dis. 2006; 10(9): 1060-2.
Steingart K R, Ng V, Henry M, Hopewell P C, Ramsay A, Cunningham J, et al. Sputum processing methods to improve the sensitivity of smear microscopy for tuberculosis: a systematic review. Lancet Infect Dis. 2006; 6(10): 664-74.
Bennedsen J, Larsen S O. Examination for tubercle bacili by fluorescence microscopy. Scand. J. Respir. Dis. 1966; 47(2): 114-20.
Trusov A, Bumgarner R, Valijev R, Chestnova R, Talevski S, Vragoterova C, et al. Comparison of Lumin LED fluorescent attachment, fluorescent microscopy and Ziehl-Neelsen for AFB diagnosis. Int J Tuberc Lung Dis. 2009; 13(7): 836-41.
Van Deun A, Chonde T M, Gumusboga M, Rienthong S. Performance and acceptability of the FluoLED Easy module for tuberculosis fluorescence microscopy. Int J Tuberc Lung Dis. 2008; 12(9): 1009-14.
WHO. Fluorescent light emitting diode microscopy for diagnosis of TB. 2010: http://www.who.int/tb/laboratory/who_policy_led_microscopy_july10.pdf.
Steingart K R, Henry M, Ng V, Hopewell P C, Ramsay A, Cunningham J, et al. Fluorescence versus conventional sputum smear microscopy for tuberculosis: a systematic review. Lancet Infect Dis. 2006; 6(9): 570-81.
Boehme C C, Nicol M P, Nabeta P, Michael J S, Gotuzzo E, Tahirli R, et al. Feasibility, diagnostic accuracy, and effectiveness of decentralised use of the Xpert MTB/RIF test for diagnosis of tuberculosis and multidrug resistance: a multicentre implementation study. Lancet. 2011; 377(9776): 1495-505.
Weyer K, Mirzayev F, Migliori G B, Van Gemert W, D'Ambrosio L, Zignol M, et al. Rapid molecular TB diagnosis: evidence, policy making and global implementation of Xpert MTB/RIF. Eur Respir J. 2013; 42(1): 252.
H. Bernhardt, J. Clasen, Flocculation of Micro-Organisms, J. Water Supply Res. Technol. AQUA 1991, 40 (2), 76-87.
L. D. Melo, E. M. Mamizuka, A. M. Carmona-Ribeiro, Antimicrobial Particles From Cationic Lipid and Polyelectrolytes, Langmuir 2010, 26 (14), 12300-12306.
U.S. Patent Application Publication No. US2010/0143883 A1, for CAPTURE OF MYCOBACTERIA LIKE MICRO-ORGANISMS, to Wilson et al., published Jun. 10, 2010.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A device for isolating one or more microorganisms from a biological sample, the device comprising a polymeric surface having one or more cationic polymers covalently grafted thereto, wherein the one or more cationic polymers have a selective affinity for the one or more microorganisms.

2. The device of claim 1, wherein the polymeric surface is hydrophobic.

3. The device of claim 1, wherein the cationic polymer comprises poly-diallyldimethyl ammonium chloride (pDADMAC).

4. The device of claim 1, wherein the polymeric surface comprises a polymer selected from the group consisting of poly(ethylene terephthalate) (PET), polystyrene (PSt), polyethylene (PE), poly(methyl methacrylate) (PMMA), or a combination thereof.

5. The device of claim 1, wherein the polymeric surface comprises a cationic monomer that has undergone UV-initiated polymerization to form a cationic-grafted polymeric surface.

6. The device of claim 1, further comprising a receptacle configured to receive the biological sample.

7. The device of claim 1, further comprising a microscope.

8. The device of claim 7, wherein the microscope is selected from the group consisting of a light microscope and a fluorescence microscope.

9. The device of claim 8, wherein the fluorescence microscope is a light-emitting diode (LED) microscope.

10. A method for isolating one or more microorganisms from a biological sample, the method comprising:
(a) providing a device comprising a polymeric surface having one or more cationic polymers covalently grafted thereto, wherein the one or more cationic polymers have a selective affinity for the one or more microorganisms;
(b) contacting the biological sample with the polymeric surface having one or more cationic polymer covalently grafted thereto to bind the one or more microorganisms to the polymeric surface;
(c) staining the bound microorganisms; and
(d) analyzing the stained bound microorganisms to determine the presence or absence of the one or more microorganisms in the biological sample.

11. The method of claim 10, further comprising diluting the biological sample.

12. The method of claim 11, wherein the diluting of the biological sample decreases viscosity and increases volume of the biological sample.

13. The method of claim 10, wherein the contacting of the biological sample with the polymeric surface having one or more cationic polymer covalently grafted thereto further comprises performing multiple mixing inversions.

14. The method of claim 10, wherein the staining of the bound microorganisms is selected from the group consisting of a Ziehl-Neelsen stain or an Auramine O stain.

15. The method of claim 10, wherein the analyzing of the stained bound microorganisms to determine the presence or absence of the one or more microorganisms in the biological sample is done by microscopy.

16. The method of claim 15, wherein the microscopy is selected from the group consisting of light microscopy, fluorescence microscopy, and LED fluorescence microscopy.

17. The method of claim 10, wherein the one or more microorganisms is a mycobacterium.

18. The method of claim 17, wherein the mycobacterium is selected from the group consisting of M. tuberculosis, M. avium, M. intracellulars, M. paratuberculosis, M. leprae, M. kansasii, M. marinum, or M. fortuitum complex.

19. A method for determining an effect of one or more therapeutic agents on the viability of one or more microorganisms from a biological sample, the method comprising:
(a) providing a device comprising a polymeric surface having one or more cationic polymers covalently grafted thereto, wherein the one or more cationic polymers have a selective affinity for the one or more microorganisms;
(b) contacting the biological sample with the polymeric surface having one or more cationic polymer covalently grafted thereto to bind the one or more microorganisms to the polymeric surface;
(c) contacting the one or more bound microorganisms with one or more therapeutic agents; and
(d) determining an effect of the one or more therapeutic agents on the viability of the one or more bound microorganisms.

20. The method of claim 19, wherein the one or more microorganisms is a mycobacterium.

21. The method of claim 20, wherein the mycobacterium is selected from the group consisting of M. tuberculosis, M. avium, M. intracellulars, M. paratuberculosis, M. leprae, M. kansasii, M. marinum, or M. fortuitum complex.

22. The method of claim 19, wherein the one or more therapeutic agents comprise an antimicrobial agent.

23. The method of claim 22, wherein the antimicrobial agent comprises an antibacterial agent.
